(19) United States
(12) Patent Application Publication
(10) Pub. No.: US 2011/0152663 A1
(43) Pub. Date: Jun. 23, 2011

(54) MEDICAL IMAGE DIAGNOSTIC APPARATUS, MEDICAL IMAGE DISPLAY DEVICE, PERSONAL INFORMATION MANAGEMENT SYSTEM

(75) Inventor: Nobuyuki KONUMA, Tochigi-ken (JP)

(73) Assignees: KABUSHIKI KAISHA TOSHIBA, Tokyo (JP); TOSHIBA MEDICAL SYSTEMS CORPORATION, Otawara-shi (JP)

(21) Appl. No.: 12/974,454

(22) Filed: Dec. 21, 2010

(30) Foreign Application Priority Data
Dec. 22, 2009 (JP) P2009-291354

(51) Int. Cl. A61B 5/05

(52) U.S. Cl. 600/407

(57) ABSTRACT

A medical image diagnostic apparatus includes an input unit to input personal information of a subject, a communication unit to communicate with a personal information managing server for storing the personal information, and a judge unit to judge whether the personal information can be transmitted to a personal information managing server. The communication unit transmits the personal information to the personal information managing server and receives personally-identifying information for identifying the personal information in response to the personal information transmitted, when the judge unit judges that the personal information can be transmitted. A memory for examination information stores the personally-identifying information.

### CROSS-REFERENCE TO RELATED APPLICATIONS

This application is based upon and claims the benefit of priority from prior Japanese Patent Application No. 2009-291354, filed Dec. 22, 2009, the entire contents of which are incorporated herein by reference.

### FIELD

Embodiments described herein relate to a medical image diagnostic apparatus, a medical image display device, and a personal information management system, and more particularly, to a medical image diagnostic apparatus, a medical image display device, and a personal information management system that are able to protect personal information of a subject.

### BACKGROUND

An ultrasonic imaging diagnostic apparatus transmits an ultrasound and receives a reflective wave formed by the difference in acoustic impedance of the tissue in the body of a subject. Recently, an ultrasonic imaging diagnostic apparatus is used in different kinds of medical fields.

The ultrasonic imaging diagnostic apparatus obtains imaging data in real time with a simple operation of being in touch with a body surface of the subject. As a result, the ultrasonic imaging diagnostic apparatus is used for different kinds of examinations, for example, heart, blood vessel, abdominal area, urinary organs, obstetrics, and gynecology, etc.

Further, in general, the ultrasonic imaging diagnostic apparatus is movable, but is not taken outside the healthcare center easily because of the size and weight of the apparatus.

Moreover, the ultrasonic imaging diagnostic apparatus can store personally-identifying information, for example, name, birth date, sex, the imaging data obtained in the examination, and doctor's comments regarding the diagnosis.

Recently, the ultrasonic imaging diagnostic apparatus is able to be miniaturized, and has a size equal to that of a laptop computer (see Jpn. Pat. App. KOKAI Publications No. 2008-253603). This portable ultrasonic imaging diagnostic apparatus is more easier carried than the conventional apparatus, and thus the apparatus has a great opportunity to be used, not only in a healthcare center, but outside the healthcare center.

However, when the apparatus is used outside the healthcare center, the personal information is exposed to the public and could be leaked.

Moreover, if the apparatus is stolen, there is a possibility that the personal information can be viewed and leaked.

Recently, a system in which a medical order for instructing a medical procedure is sent to the mobile terminal device by wireless and received by the medical staff is popular. Further, a doctor's car that has an ultrasonic imaging diagnostic apparatus that is bigger than the portable apparatus described above, an X-ray diagnosis apparatus, or an X-ray computed tomography apparatus are able to be used away from the site of the healthcare center. Therefore leaking of personal information is more and more of a problem.

### BRIEF DESCRIPTION OF THE DRAWINGS

Aspects of this disclosure will become apparent upon reading the following detailed description and upon reference to the accompanying drawings. The description and the associated drawings are provided to illustrate embodiments of the invention and do not limit the scope of the invention, wherein:

FIG. 1 is a block diagram showing a structure of a personal information protecting system according to a first embodiment;

FIG. 2 is a flowchart showing a process executed by the personal information protecting system during the examination according to the first embodiment;

FIG. 3 is a flowchart showing a process executed by the personal information protecting system for outputting the examination information in response to the input of the personal information during the examination according to the first embodiment;

FIG. 4 is a flowchart showing a process executed by the personal information protecting system for outputting the examination information in response to the input of the personally-identifying information during the examination according to the first embodiment;

FIG. 5 is a flowchart showing a process executed by the personal information protecting system for transferring the examination information from the ultrasonic imaging diagnostic apparatus to the imaging server that connects the network in the healthcare center according to the first embodiment;

FIG. 6 is a flowchart showing a process executed by the personal information protecting system for transferring the examination information from the imaging server that connects the network in the healthcare center to the ultrasonic imaging diagnostic apparatus according to the first embodiment;

FIG. 7 is a block diagram showing a structure of a personal information protecting system according to a second embodiment;

FIG. 8 is a flowchart showing a process executed by the personal information protecting system during the examination according to the second embodiment;

FIG. 9 is a flowchart showing a process executed by the personal information protecting system for outputting the examination information in response to the input of the personal information during the examination according to the second embodiment; and

FIG. 10 is a flowchart showing a process executed by the personal information protecting system for outputting the examination information in response to the input of the personally-identifying information during the examination according to the second embodiment.

### DETAILED DESCRIPTION

Embodiments described herein solve the problems described above, and provide a medical image diagnostic apparatus, a medical image display device, and a personal information management system that are able to prevent the leaking of personal information.

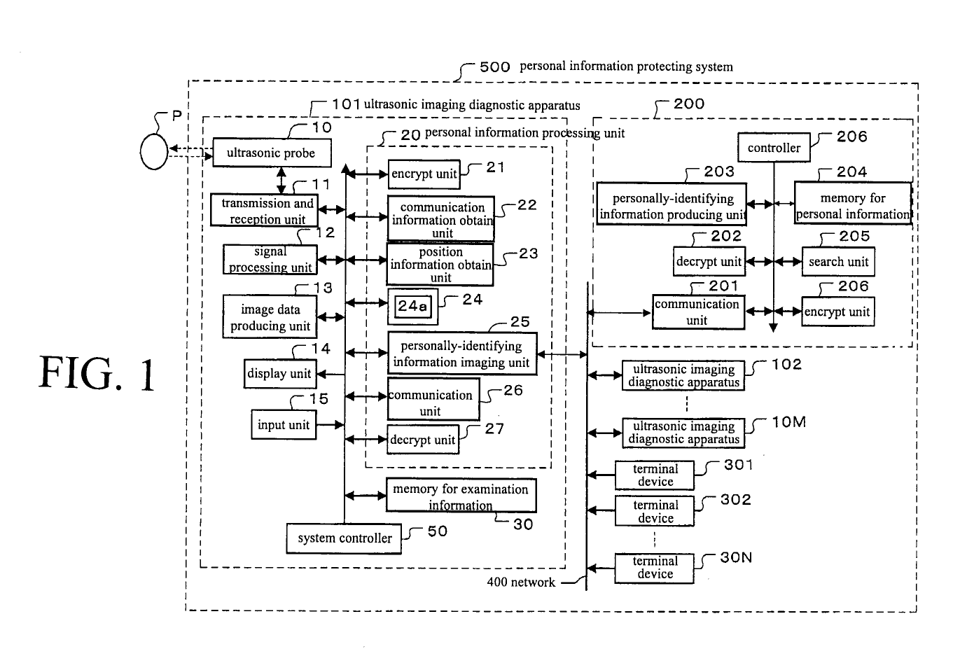

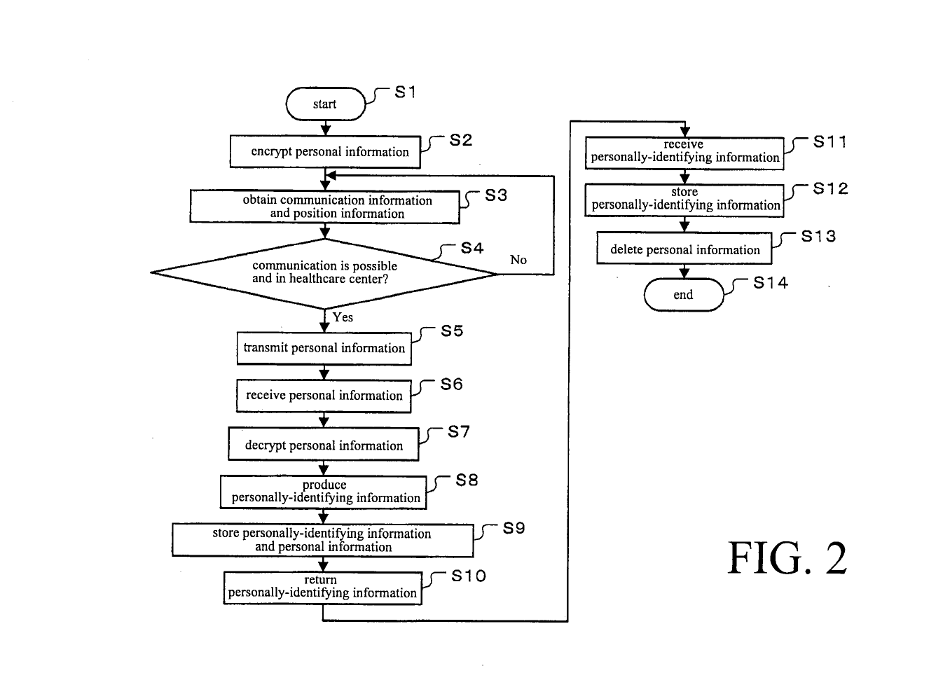

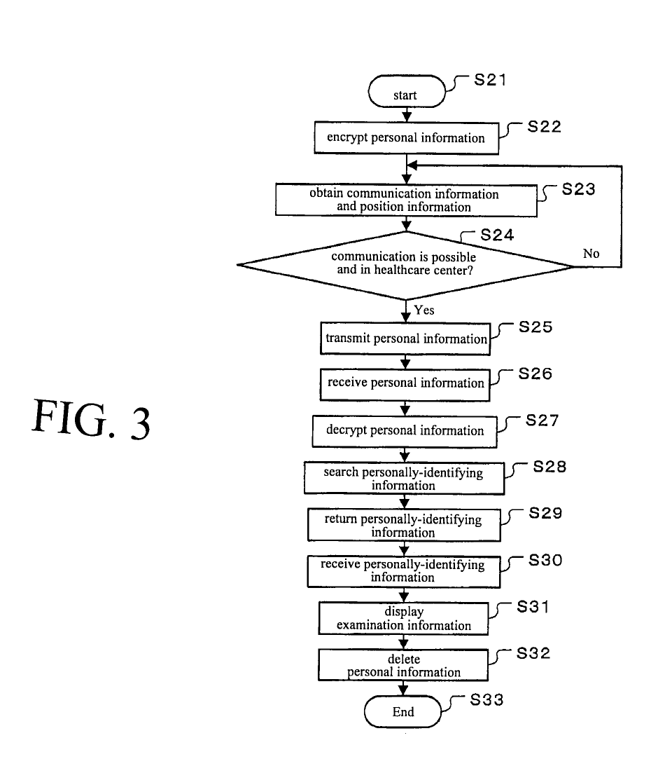

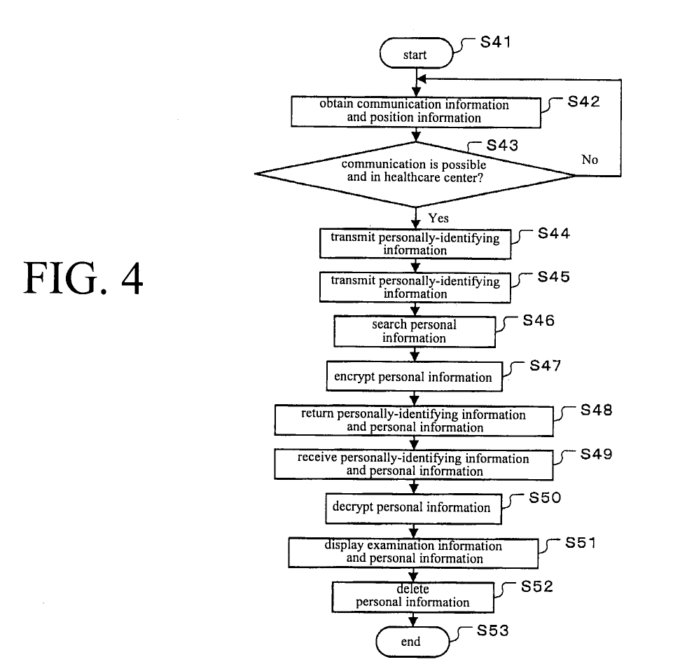

Hereinafter, an embodiment of the personal information protecting system used with an ultrasonic imaging diagnostic apparatus is described with reference to FIG. 1, FIG. 2, FIG. 3, and FIG. 4.

FIG. 1 is a block diagram showing a structure of a personal information protecting system 500 according to a first embodiment.

The personal information protecting system 500 has one or more ultrasonic imaging diagnostic apparatuses 101-10M. Each ultrasonic imaging diagnostic apparatus 101-10M is portable and arranged in the healthcare center, for example, a hospital, and is able to examine the subject P.

The personal information protecting system 500 has a personal information managing server 200. The personal information managing server 200 connects each ultrasonic imaging diagnostic apparatus 101-10M through network 400, and generates the personally-identifying information for identifying the personal information, for example, the name of the subject P, birth date, sex, and so on, and stores the personal information.

In addition, the personal information protecting system 500 has one or more terminal devices 301-30N. Each terminal device 301-30N is arranged in the healthcare center and connects to each ultrasonic imaging diagnostic apparatus 101-10M, and has an inputting unit for inputting the personal information of the subject P for accepting the examination by the ultrasonic imaging diagnostic apparatus 101-10M.

First, the ultrasonic imaging diagnostic apparatus 101 will be described.

Each ultrasonic imaging diagnostic apparatus 102-10M is similar to the ultrasonic imaging diagnostic apparatus 101, and the description of each individual apparatus will be skipped.

The ultrasonic imaging diagnostic apparatus 101 includes an ultrasonic probe 10 that transmits an ultrasound to the subject P and receives the reflective wave transmission; a transmission and reception unit 11 to transmit the ultrasound generating signal to the ultrasonic probe 10 and to receive the reflective wave signal; a signal processing unit 12 to process the reflective wave signal and to generate B-mode data and Doppler data; and an image data producing unit 13 to produce the B-mode image data and the Doppler image data based on the B-mode data and the Doppler data.

Further, the ultrasonic imaging diagnostic apparatus 101 has a display unit 14 to display the image data produced by the image data producing unit 13; an input unit 15 to input comment information for the image data produced in the image data producing unit 13; and a personal information processing unit 20 to execute a process for protecting the personal information input by the input unit 15.

Additionally, the ultrasonic imaging diagnostic apparatus 101 has a memory for examination information 30 to store the examination information, including the image data and the comment information for the image data and the personally-identifying information related to this examination that is generated in the personal information managing server 200; and a system controller 50 to control the above-described units.

The ultrasonic probe 10 transmits the ultrasound to the subject P, with the head of the ultrasonic probe 10 being in touch with the body surface of the subject P. The probe 10 has, for example, a plurality of piezoelectric vibrators arranged in one dimension.

Each piezoelectric vibrator converts the ultrasound generating signal from the transmission and reception unit 11 to ultrasound, transmits the ultrasound to the subject P, and receives the ultrasound reflected from the subject P and generates the reflective wave signal. The piezoelectric vibrator outputs the reflective wave signal to the transmission and reception unit 11.

The transmission and reception unit 11 produces the ultrasound generating signal received from the ultrasonic probe 10.

Further, the transmission and reception unit 11 phase rectifies and adds the reflective wave signal obtained by a plurality of channels and outputs the signal to the signal processing unit 12.

The signal processing unit 12 executes a process on the signal output from the transmission and reception unit 11, produces the B-mode data and the Doppler data, and outputs the B-mode data and the Doppler data to the image data producing unit 13.

The image data producing unit 13 produces the image data, for example, the B-mode image data and the Doppler image data, by converting the B-mode data and the Doppler data.

Further, the image data producing unit 13 outputs the image data to the display unit 14 and the memory for examination information 30.

The display unit 14 has a liquid crystal panel or CRT (cathode-ray tube) and displays the image data produced by the image data producing unit 13 and the examination information stored in the memory for examination information 30.

The input unit 15 has input devices, for example, buttons, switches, a keyboard, a trackball, a mouse, etc.

The input unit 15 is configured to input imaging conditions (gain, FOV (field of view) depth, transmit frequency, convergent point, pulse recurrence frequency, producing mode, and so on) to produce the image data.

The input unit 15 inputs the personal information of the subject P undergoing the examination.

The input unit 15 inputs the comment information for the image data produced by the image data producing unit 13.

Further, the input unit 15 inputs the personal information and the personally-identifying information stored in the memory for examination information 30 of the subject P undergoing the examination to the display unit 14 to display the examination information stored in the memory for examination information 30.

The personal information processing unit 20 has an encrypt unit 21 to encrypt the personal information sent by each terminal device 301-30N or input by the input unit 15; a communication information obtain unit 22 to obtain the communication information for judging whether communication with the personal information managing server 200 is possible or impossible; and a position information obtain unit 23 to obtain position information of the ultrasonic imaging diagnostic apparatus 101 by using the RFID (Radio Frequency Identification) or wireless LAN (Local Area Network).

The personal information processing unit 20 has a judge unit 24 to judge whether the personal information encrypted by the encrypt unit 21 can be sent to the personal information managing server 200, and a memory 24a to store the personal information during the time the memory for examination information 30 stores the personally-identifying information produced in the personal information managing server 200 for identifying the personal information when the judge unit 24 judges that communication with the personal information managing server is possible.

Additionally, the personal information processing unit 20 has a personally-identifying information managing unit 25 to relate the examination information stored in the memory for examination information 30 to the personally-identifying information produced by the personal information managing server 200, wherein the personally-identifying information managing unit 25 has a memory to store the personally-identifying information.

Additionally, the personal information processing unit 20 has a communication unit 26 to communicate with the personal information managing server 200 and the terminal devices 301-30N through the network 400, and a decrypt unit 27 to decrypt the encrypted personal information returned to the communication unit 26 through the network 400 from the personal information managing server 200 in response to the personally-identifying information input by the input unit 15.

Further, the communication unit 26 receives the personal information transmitted by the terminal devices 301-30N through the network 400 when receiving the examination order.

Further, the communication unit 26 transmits the personal information encrypted by the encrypt unit 21 to the personal information managing server 200 through the network 400, and receives the personally-identifying information returned through the network 400 from the personal information managing server 200 in response to the transmission.

Additionally, the input unit 15 transmits the personally-identifying information stored in the memory for the examination information 30 to the personal information managing server 200 through the network 400, and receives the encrypted personal information returned from the personal information managing server 200 through the network 400.

The memory for examination information 30 has a hard disc and stores the image data produced by the image data producing unit 13 for each examination, the comment information input by the input unit 15 regarding the image data, and the personally-identifying information related to the examination information and produced by the personal information managing server 200.

The system controller 50 has a CPU and a memory circuit and controls the transmission and reception unit 11, the signal processing unit 12, the image data producing unit 13, the display unit 14, the personal information processing unit 20, the memory for examination information 30, and the overall system, based on the personal information transmitted from the terminal device 301-30N and the input information, for example, the personal information input by the input unit 15 and the imaging condition.

Next, the personal information managing server 200 will be described.

The personal information managing server 200 has a communication unit 201 to receive the encrypted personal information and the personally-identifying information from the communication unit 26 of the personal information processing unit 20 in the ultrasonic imaging diagnostic apparatus 101, a decrypt unit 202 to decrypt the personal information received by the communication unit 201, and a personally-identifying information producing unit 203 to produce the personally-identifying information to identify the personal information decrypted by the decrypt unit 202.

Further, the personal information managing server 200 has a memory for personal information 204 to store the personally-identifying information produced by the personally-identifying information producing unit 203 with the personal information decrypted by the decrypt unit 202, and a search unit 205 to search the personal information related to the personally-identifying information decrypted by the decrypt unit 202 and received by the communication unit 201, in the personally-identifying information and the personal information stored in the memory for the personal information 204.

Additionally, the personal information managing server 200 has an encrypt unit 206 to encrypt the personal information searched by the search unit 205, and a controller 207 to control the communication unit 201, the decrypt unit 202, the personally-identifying information producing unit 203, the memory for personal information 204, the search unit 205, and the encrypt unit 206.

Further, the communication unit 201 returns the personally-identifying information produced by the personally-identifying information producing unit 203 through the network 400 in response to the transmission of the personal information input in the reception of the examination order.

Further, the communication unit 201 returns the personally-identifying information to identify the personal information searched by the search unit 205 through the network 400 in response to the transmission of the personal information input for displaying the examination information.

Additionally, the communication unit 201 returns the personal information encrypted by the encrypt unit 206 and the personally-identifying information to identify the personal information through the network 400 in response to the transmission of the personally-identifying information input for displaying the examination information.

In this way, the personal information input by each terminal device 301-30N or the input unit 15 can be stored in the personal information managing server 200 outside the ultrasonic imaging diagnostic apparatus 101.

Hereinafter, an example of a process executed by the personal information protecting system 500 will be described with reference to FIG. 1 , FIG. 2, and FIG. 3.

FIG. 2 is a flowchart showing a process executed by the personal information protecting system 500 during the examination.

FIG. 3 and FIG. 4 are flowcharts showing a process executed by the personal information protecting system 500 for outputting the examination information.

FIG. 3 is a flowchart showing a process executed by of the personal information protecting system 500 for outputting the examination information in response to the input of the personal information, and FIG. 4 is a flowchart showing a process executed by the personal information protecting system 500 for outputting the examination information in response to the input of the personally-identifying information.

At first, the process executed by the personal information protecting system 500 during the examination will be described.

In FIG. 2, for the examination of the subject P using the ultrasonic imaging diagnostic apparatus 101 when the personal information of the subject P is input by the input unit 15 of the ultrasonic imaging diagnostic apparatus 101, the personal information protecting system 500 starts the examination process. (Step S1)

In step S2, the system controller 50 of the ultrasonic imaging diagnostic apparatus 101 sends an order for the start of the examination to the memory for examination information 30. The encrypt unit 21 of the personal information processing unit 20 encrypts the personal information inputted by the input unit 15 and outputs the encrypted personal information to the judge unit 24.

In step S3, the communication information obtain unit 22 and the communication information taking unit 23 obtain the communication information and the position information at constant time intervals and output the communication information and the position information to the judge unit 24.

After the judge unit 24 stores the personal information output from the encrypt unit 21 in the memory 24a, the judge unit 24 judges whether the personal information stored in the memory 24a can be sent to the personal information managing server 200, based on the communication information output from the communication information obtain unit 22 and the position information obtain unit 23.

When RFID and wireless LAN is used, the judge unit 24 judges that the ultrasonic imaging diagnostic apparatus 101 is outside the healthcare center when the access point is not found in the healthcare center.

Further, when the ultrasonic imaging diagnostic apparatus 101 can communicate with the personal information managing server 200 and the ultrasonic imaging diagnostic apparatus 101 is in the healthcare center (Yes in Step S4), the process proceeds to Step S5.

Further, when the ultrasonic imaging diagnostic apparatus 101 cannot communicate with the personal information managing server 200 or the ultrasonic imaging diagnostic apparatus 101 is outside the healthcare center (No in Step S4), the process proceeds to Step S3.

When the ultrasonic imaging diagnostic apparatus 101 moves from outside the healthcare center to inside the healthcare center, the ultrasonic imaging diagnostic apparatus 101 tries to connect to the network 400, and when the ultrasonic imaging diagnostic apparatus 101 can communicate with the personal information managing server 200, the process moves to Step S5.

After “Yes in Step S4”, the judge unit 24 stores the personal information in the memory 24a and outputs the personal information to the communication unit 26.

In step S5, the communication unit 26 transmits the personal information output by the judge unit 24 to the personal information managing server 200 through the network 400.

The communication unit 201 of the personal information managing server 200 receives the personal information transmitted by the ultrasonic imaging diagnostic apparatus 101 and outputs the received personal information to the decrypt unit 202.

In step S7, the decrypt unit 202 decrypts the personal information output by the communication unit 201, and outputs the decrypted personal information to the personally-identifying information producing unit 203.

In step S8, the personally-identifying information producing unit 203 produces the personally-identifying information to identify the personal information output by decrypt unit 202, and outputs the personally-identifying information to the communication unit 201, and outputs the personally-identifying information and the personal information decrypted by the decrypt unit 202 and identified by the personally-identifying information.

In step S9, the memory for personal information 204 stores the personally-identifying information and the personal information output by the personally-identifying information producing unit 203.

Thus, the leaking of the personal information from the ultrasonic imaging diagnostic apparatus 101 inside and outside the healthcare center can be prevented by storing the personal information input by input unit 15 to the personal information managing server 200, which is outside the ultrasonic imaging diagnostic apparatus 101.

Further, when the ultrasonic imaging diagnostic apparatus 101 is carried outside the healthcare center, the system can prevent the personal information from being publicly exposed and being leaked.

Further, when the ultrasonic imaging diagnostic apparatus 101 is carried outside the healthcare center and is stolen, the system can prevent the personal information from being viewed and being leaked.

In step S10, the communication unit 201 returns the personally-identifying information output by the personally-identifying information producing unit 203.

In step S11, the communication unit 26 of the ultrasonic imaging diagnostic apparatus 101 receives the personally-identifying information returned from the personal information managing server 200 through the network 400, and outputs the received personally-identifying information to the personally-identifying information managing unit 25.

When the personal information is input by the input unit 15 and the operation for starting the examination of the subject P that has the personal information begins, the signal processing unit 12 processes the received signal output by the transmission and reception unit 11 and produces the B-mode data and outputs the B-mode data to the image data producing unit 13.

The image data producing unit 13 produces the B-mode image data based on the B-mode data output by the signal processing unit 12, and the display unit 14 displays the B-mode image data in real time.

Further when the display unit 14 displays the B-mode image data that is helpful for the examination, and a stop operation is executed by the input unit 15, the image data producing unit 13 stores the examination information including the B-mode image data to the memory for examination information 30.

The personally-identifying information managing unit 25 displays the personally-identifying information output by the communication unit 26 on the display unit 14.

In step S12, the communication unit 26 stores the personally-identifying information related to the examination information of the subject P that has the personal information that is identified by the personally-identifying information.

When the ultrasonic imaging diagnostic apparatus 101 is carried outside the healthcare center and is used in the examination, and after the examination, the ultrasonic imaging diagnostic apparatus 101 is returned inside the healthcare center, the examination information is stored in the ultrasonic imaging diagnostic apparatus 101, the display unit 14 displays the examination information output by the communication unit 26, and the personally-identifying information is stored related to the examination information in the memory for examination information 30.

Further, when the ultrasonic imaging diagnostic apparatus 101 is used in the examination, the personally-identifying information output by communication unit 26 is stored in the memory of the ultrasonic imaging diagnostic apparatus 101, and the examination information of the subject P that has the personal information identified by the personally-identifying information and the personally-identifying information stored related to the stored examination information in the memory is stored.

Thus, the examination information is stored in the ultrasonic imaging diagnostic apparatus 101 and the examination information can be identified by storing the personally-identifying information related to the examination information.

Further, when the ultrasonic imaging diagnostic apparatus 101 is carried outside the healthcare center, because the personal information cannot be identified, even if the personally-identifying information is viewed, the personal information can be prevented from being leaked.

Additionally, when the ultrasonic imaging diagnostic apparatus 101 is carried outside the healthcare center and is leaked, because the personal information cannot be identified, even if the personally-identifying information is viewed, the personal information can be prevented from being leaked.

In step S13, after the personally-identifying information is stored, the personal information stored in the memory 24a is deleted.

Thus, after the personally-identifying information that identifies the personal information and produced by the personal information managing server 200 is stored in the memory 24a for examination information, the personal information can be prevented from being viewed by an outsider other than the operator, despite the ultrasonic apparatus being inside and outside the healthcare center.

In step S14, after the personal information is deleted, the personal information protecting system 500 ends the process of examination.

The personal information input by the input unit 15 is transmitted to the personal information managing server 200 before the end of the examination.

In another embodiment, the personal information is transmitted to the personal information managing server 200 at the beginning of the examination.

Next, a method executed by the personal information protecting system 500 for outputting the examination information in response to the input of the personal information is described.

FIG. 3 is a flowchart showing a process executed by the personal information protecting system 500 for outputting the examination information in response to the input of the personal information.

After the examination information and the personally-identifying information are stored by the memory for examination information 30 in the ultrasonic imaging diagnostic apparatus 101, when the personal information of the subject P is input by the input unit 15 of the ultrasonic imaging diagnostic apparatus 101, the personal information protecting system 500 starts the examination process. (Step S21.)

The system controller 50 of the ultrasonic imaging diagnostic apparatus 101 gives an order to output the examination information to the display unit 14, the personal information processing unit 20, and the memory for examination information 30.

In step S22, the encrypt unit 21 of personal information processing unit 20 encrypts the personal information input by the input unit 15 and outputs the encrypt information to the judge unit 24.

In step S23, the communication information obtain unit 22 and the position information obtain unit 23 obtain the communication information and the position information at constant time intervals and output the communication information and the position information to the judge unit 24.

After the judge unit 24 stores the personal information output from the encrypt unit 21 in the memory 24a, the judge unit 24 judges whether the personal information output by the encrypt unit 21 can be sent to the personal information managing server 200 based on the communication information output from the communication information obtain unit 22 and the position information obtain unit 23.

When the ultrasonic imaging diagnostic apparatus 101 can communicate with the personal information managing server 200 and the ultrasonic imaging diagnostic apparatus 101 is in the healthcare center (Yes in Step S24), the process proceeds to Step S25.

Further, when the ultrasonic imaging diagnostic apparatus 101 cannot communicate with the personal information managing server 200 or the ultrasonic imaging diagnostic apparatus 101 is outside the healthcare center (No in Step S24), the process proceeds to Step S23.

After Step S24, the judge unit 24 stores the personal information and outputs the personal information to the communication unit 26.

In step S25, the communication unit 26 transmits the personal information output by the judge unit 24 to the personal information managing server 200 through the network 400.

In step S26, the communication unit 201 of the personal information managing server 200 receives the personal information transmitted by the ultrasonic imaging diagnostic apparatus 101 through the network 400.

The communication unit 201 outputs the personal information received by the ultrasonic imaging diagnostic apparatus 101.

In step S27, the decrypt unit 202 decrypts the personal information received by the communication unit 201 and outputs the decrypted personal information to the search unit 205.

In step S28, the search unit 205 searches the personally-identifying information related to the personal information output by the decrypt unit 202 in the personally-identifying information and the personal information stored by the memory for personal information 204 and outputs the searched personally-identifying information.

In step S29, the communication unit 201 returns the personally-identifying information output by the search unit 205 through the network 400.

In step S30, the communication unit 26 of the ultrasonic imaging diagnostic apparatus 101 receives the personally-identifying information returned from the personal information managing server 200 through the network 400 and outputs the received personally-identifying information to the personally-identifying information managing unit 25.

In step S31, the personally-identifying information managing unit 25 reads the examination information related to the personally-identifying information and output by communication unit 26, and displays the read examination information.

In step S32, after the viewing of the examination information displayed by the display unit 14, when an operation of ending the viewing is executed by the input unit 15, the judge unit 24 deletes the personal information stored in the memory 24a.

Thus, after the personally-identifying information that identifies the personal information and produced by the personal information managing server 200 is stored in the ultrasonic imaging diagnostic apparatus 101, the personal information can be prevented from being viewed by an outsider except the operator despite the ultrasonic imaging apparatus being inside and outside the healthcare center.

In step S33, and when the system controller 50 gives an order to stop transmission to the display unit 14, the personal information processing unit 20, and the memory for examination information 30, the personal information protecting system 500 stops the process of examination.

The examination information can be output by inputting the personally-identifying information except for inputting the personal information.

In what follows, a process executed by the personal information protecting system 500 when the examination information is output in response to inputting the personally-identifying information will be described.

FIG. 4 is a flowchart showing a method executed by the personal information protecting system 500 for outputting the examination information, in response to the input of the personally-identifying information during the examination.

In Step S12 in FIG. 2, when the personally-identifying information stored in the memory for examination information 30 is displayed on the display 14, for example, the personally-identifying information for identifying the personal information of the patient is recorded in the medical record.

In step S41, after the examination information and the personally-identifying information are stored in the memory for examination information 30 of the ultrasonic imaging diagnostic apparatus 101, when the personally-identifying information is input by the input unit 15, the personal information protecting system 500 starts to output the examination information.

The display 14 displays the view of the personally-identifying information stored in the memory for examination information 30, and one of the personally-identifying information is selected by the input unit 15 in the view displayed.

The system controller 50 of the ultrasonic imaging diagnostic apparatus 101 gives an order to output the examination information to the display unit 14, the personal information processing unit 20, and the memory for examination information 30.

In step S42, the communication information obtain unit 22 and the communication information taking unit 23 obtain the communication information and the position information at constant time intervals and output the communication information and the position information to the judge unit 24.

After the personally-identifying information input by the input unit 15 is stored in the memory 24a, the judge unit 24 judges whether the personally-identifying information input can be sent to the personal information managing server 200 based on the communication information output from the communication information obtain unit 22 and the position information output from the position information obtain unit 23.

When the ultrasonic imaging diagnostic apparatus 101 can communicate with the personal information managing server 200 and the ultrasonic imaging diagnostic apparatus 101 is in the healthcare center (Yes in Step S43), the process proceeds to Step S44.

When the ultrasonic imaging diagnostic apparatus 101 cannot communicate with the personal information managing server 200 or the ultrasonic imaging diagnostic apparatus 101 is outside the healthcare center (No in Step S43), the process returns to Step S42.

After “Yes in Step S43”, the judge unit 24 stores the personally-identifying information in the memory 24a and outputs the personally-identifying information to the communication unit 26.

In step S44, the communication unit 26 transmits the personally-identifying information output by the judge unit 24 to the personal information managing server 200 through the network 400.

In step S45, the communication unit 201 of the personal information managing server 200 receives the personally-identifying information transmitted by the ultrasonic imaging diagnostic apparatus 101 through the network 400.

The communication unit 201 outputs the personally-identifying information received by the ultrasonic imaging diagnostic apparatus 101.

In step S46, the search unit 205 searches the personal information related to the personally-identifying information output by the communication unit 201 in the personally-identifying information and the personal information stored by the memory for personal information 204, and outputs the searched personal information and the personally-identifying information related to the personal information to the encrypt unit 206.

In step S47, the encrypt unit 206 encrypts the personal information output by the search unit 205 and outputs the encrypted personal information and the personally-identifying information related to the personal information to the communication unit 201.

In step S48, the communication unit 201 returns the encrypted personal information and the personally-identifying information output by the encrypt unit 206.

In step S49, the communication unit 26 of the ultrasonic imaging diagnostic apparatus 101 receives the personal information and the personally-identifying information returned from the personal information managing server 200 through the network 400 and outputs the received personal information and the personally-identifying information to the decrypt unit 27.

In step S50, the decrypt unit 27 decrypts the personal information output by the communication unit 26, and outputs the decrypted personal information and the personal information related to the personal information to the personally-identifying information managing unit 25.

In step S51, the personally-identifying information managing unit 25 reads the examination information related to the personally-identifying information output from the decrypt unit 27 and displays the read examination information.

In step S52, after the viewing of the examination information and the personal information displayed by the display unit 14, when an operation ending viewing is executed by the input unit 15, the judge unit 24 deletes the personally-identifying information stored in the memory 24a.

In step S53, when the system controller 50 gives an order to stop transmission to the display unit 14, the personal information processing unit 20, and the memory for examination information 30, the personal information protecting system 500 stops the process of examination.

Additionally, an example of the process executed by the personal information protecting system 500 will be described when the examination information of the examination executed for a specific patient outside of the healthcare center is brought back, and when the examination information of the examination already executed for the patient is taken outside the healthcare center for a home visit of the specific patient.

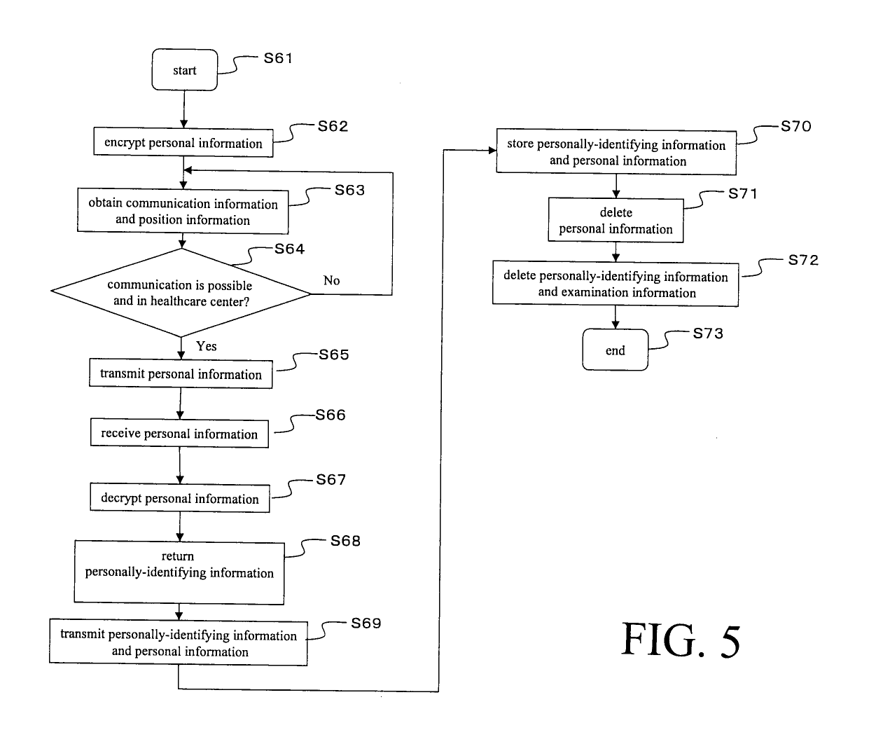

FIG. 5 is a flowchart showing a method executed by the personal information protecting system 500 for transferring the examination information of the particular patient inputted by the input unit 15 from the ultrasonic imaging diagnostic apparatus 101 to an imaging server that connects the network 400 in the healthcare center.

In step S61, when the examination information of the examination executed by the medical staff for a specific patient outside of the healthcare center is brought back and the personal information to be transmitted to the imaging server that connects the network 400 in the healthcare center is input by the input unit 15, the personal information protecting system 500 starts the operation to output the examination information of the specific patient from the memory for examination information 30 of the ultrasonic imaging diagnostic apparatus 101 to the imaging server.

The system controller 50 of the ultrasonic imaging diagnostic apparatus 101 gives an order to output the personal information to the personal information processing unit 20.

In step S62, the encrypt unit 21 of the personal information processing unit 20 encrypts the personal information input by the input unit 15 and outputs the encrypted personal information to the judge unit 24.

In step S63, the communication information obtain unit 22 and the position information obtain unit 23 obtain the communication information and the position information at constant time intervals and output the communication information and the position information to the judge unit 24.

After the judge unit 24 stores the personal information output from the encrypt unit 21 in the memory 24a, the judge unit 24 judges whether the personal information output by the encrypt unit 21 can be sent to the personal information managing server 200 based on the communication information output from the communication information obtain unit 22 and the position information obtain unit 23.

Further, when the ultrasonic imaging diagnostic apparatus 101 can communicate with the personal information managing server 200 and the ultrasonic imaging diagnostic apparatus 101 is in the healthcare center (Yes in Step S64), the process proceeds to Step S65.

When the ultrasonic imaging diagnostic apparatus 101 cannot communicate with the personal information managing server 200 or the ultrasonic imaging diagnostic apparatus 101 is outside the healthcare center (No in Step S64), the process returns to Step S63.

After Step S64, the judge unit 24 stores the personal information and outputs the personal information to the communication unit 26.

In step S65, the communication unit 26 transmits the personal information output by the judge unit 24 to the personal information managing server 200 through the network 400.

In step S66, the communication unit 201 of the personal information managing server 200 receives the personal information transmitted by the ultrasonic imaging diagnostic apparatus 101 through the network 400.

The communication unit 201 outputs the personal information received by the ultrasonic imaging diagnostic apparatus 101.

In step S67, the decrypt unit 202 decrypts the personal information received by the communication unit 201 and outputs the decrypted personal information to the search unit 205.

The search unit 205 searches the personally-identifying information related to the personal information output by the decrypt unit 202 in the personally-identifying information and the personal information stored by the memory for personal information 204, and outputs the searched personally-identifying information to the communication unit 201.

In step S68, the communication unit 201 returns the personally-identifying information output by the search unit 205 through the network 400.

The communication unit 26 of the ultrasonic imaging diagnostic apparatus 101 receives the personally-identifying information returned from the personal information managing server 200 through the network 400, and outputs the received personally-identifying information to the personally-identifying information managing unit 25.

In step S69, the personally-identifying information managing unit 25 reads the examination information related to the personally-identifying information and output by the communication unit 26, and outputs the read examination information with the examination information to the communication unit 26.

The communication unit 26 outputs the input personally-identifying information and the examination information to the communication unit 201.

In step S70, the communication unit 201 outputs the input personally-identifying information and the examination information to the imaging server connected to the network 400 in the healthcare center, and the imaging server stores the personally-identifying information and the examination information.

In step S71, after the imaging server finishes storing the personally-identifying information and the examination information, the personal information stored in the memory 24a is deleted by the judge unit 24.

In step S72, the system processing unit 50 gives an order to delete the examination information and the personally-identifying information in the memory for examination information 30, and the memory for examination information 30 deletes the examination information and the personally-identifying information.

In step S73, when the system controller 50 gives an order to stop the operation to the memory for examination information 30, the personal information protecting system 500 stops outputting the examination information and the personally-identifying information from the memory for examination information 30 to the imaging server.

Next, when the examination information of the examination already executed for the patient is taken outside the healthcare center for a home visit of the specific patient, the method executed by the personal information protecting system 500 will be described.

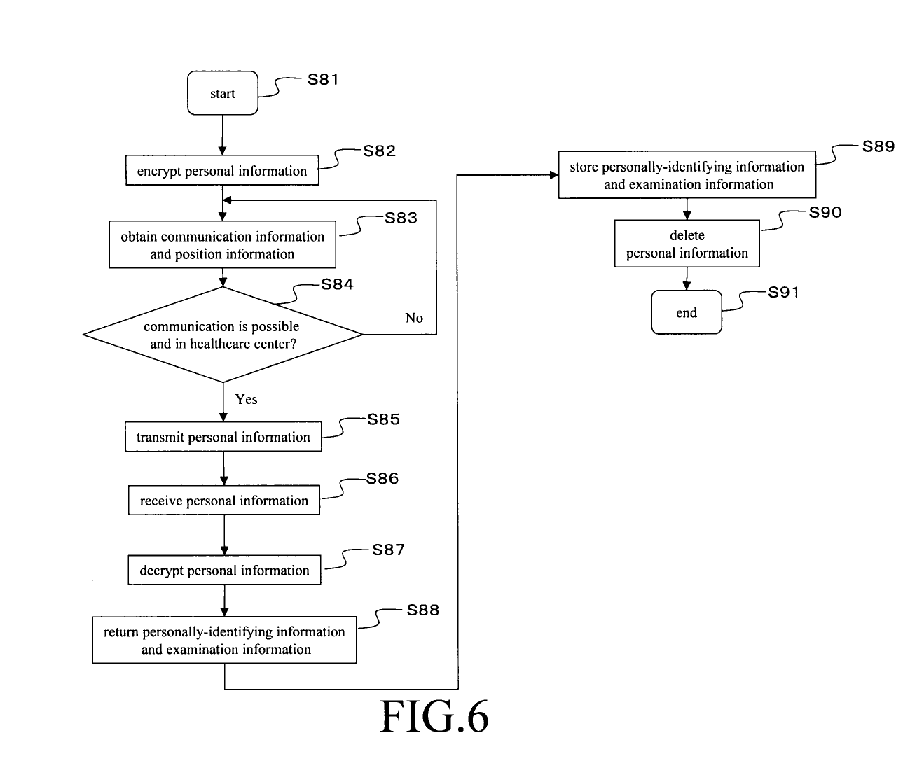

FIG. 6 is a flowchart showing a process executed by the personal information protecting system 500 for transferring the examination information of the particular patient inputted by the input unit 15 from the imaging server that connects the network 400 in the healthcare center to the memory for examination information 30 in the ultrasonic imaging diagnostic apparatus 101.

In step S81, when the examination information of the examination already executed for the patient is taken outside the healthcare center for a home visit of the specific patient, and the personal information to be taken outside the healthcare center is input by the input unit 15, the personal information protecting system 500 starts the operation to output the examination information of the specific patient from the imaging server to the memory for examination information 30 of the ultrasonic imaging diagnostic apparatus 101.

The system controller 50 of the ultrasonic imaging diagnostic apparatus 101 gives an order to output the personal information to the personal information processing unit 20.

In step S82, the encrypt unit 21 of the personal information processing unit 20 encrypts the personal information input by the input unit 15 and outputs the encrypted personal information to the judge unit 24.

In step S83, the communication information obtain unit 22 and the position information obtain unit 23 obtain the communication information and the position information at constant time intervals and output the communication information and the position information to the judge unit 24.

After the judge unit 24 stores the personal information output from the encrypt unit 21 in the memory 24a, the judge unit 24 judges whether the personal information output by the encrypt unit 21 can be sent to the personal information managing server 200 based on the communication information output from the communication information obtain unit 22 and the position information obtain unit 23.

When the ultrasonic imaging diagnostic apparatus 101 can communicate with the personal information managing server 200 and the ultrasonic imaging diagnostic apparatus 101 is in the healthcare center (Yes in Step S84), the process proceeds to Step S85.

Further, when the ultrasonic imaging diagnostic apparatus 101 cannot communicate with the personal information managing server 200 or the ultrasonic imaging diagnostic apparatus 101 is outside the healthcare center (No in Step S84), the process returns to Step S83.

After Step S84, the judge unit 24 stores the personal information and outputs the personal information to the communication unit 26.

In step S85, the communication unit 26 transmits the personal information output by the judge unit 24 to the personal information managing server 200 through the network 400.

In step S86, the communication unit 201 of the personal information managing server 200 receives the personal information transmitted by the ultrasonic imaging diagnostic apparatus 101 through the network 400.

The communication unit 201 outputs the personal information received by the ultrasonic imaging diagnostic apparatus 101.

In step S87, the decrypt unit 202 decrypts the personal information received by the communication unit 201 and outputs the decrypted personal information to the search unit 205.

The search unit 205 searches the personally-identifying information related to the personal information output by the decrypt unit 202 in the personally-identifying information and the personal information stored by the memory for personal information 204, and outputs the searched personally-identifying information to the imaging server.

The imaging server outputs the examination information stored with the personally-identifying information related to the examination information based on the input personally-identifying information.

In step S88, the communication unit 201 returns the output personally-identifying information and the examination information to the ultrasonic imaging diagnostic apparatus 101 through the network 400.

In step S89, the communication unit 26 of the ultrasonic imaging diagnostic apparatus 101 receives the personally-identifying information and the examination information returned to the personal information managing server 200 and outputs the personally-identifying information and the examination information to the memory for examination information 30, and the memory for examination information 30 relates the input examination information to the personally-identifying information and stores the examination information and the personally-identifying information.

In step S90, when the examination information and the personally-identifying information are stored in the memory for examination information 30, the judge unit 24 deletes the personal information stored in the memory 24a.

In step S91, when the system controller 50 gives an order to stop transmission to the memory for examination information 30, the personal information protecting system 500 stops the process of outputting the examination information of the specific patient to the memory for examination information 30 of the ultrasonic imaging diagnostic apparatus 101 from the imaging server.

According to the embodiment described above, the personal information for the examination input by a terminal device 301-30N or the input unit 15 can be stored in the personal information managing server 200 outside the ultrasonic imaging diagnostic apparatus 101.

Herewith, when the ultrasonic imaging diagnostic apparatus 101 is carried outside the healthcare center, the system can prevent the personal information from being publicly exposed and being leaked.

Further, when the ultrasonic imaging diagnostic apparatus 101 is carried outside the healthcare center and is stolen, the system can prevent the personal information from being viewed and being leaked.

From the time the memory for examination information 30 stores the personally-identifying information produced in the personal information managing server 200 for identifying the personal information, the personal information input for the examination can be encrypted and stored in the ultrasonic imaging diagnostic apparatus 101.

Additionally, the personally-identifying information can be related to the examination and be stored in the ultrasonic imaging diagnostic apparatus 101.

Herewith the examination information is stored in the ultrasonic imaging diagnostic apparatus 101 and the examination information can be identified by storing the personally-identifying information related to the examination information.

Further, when the ultrasonic imaging diagnostic apparatus 101 is carried outside the healthcare center, because the personal information cannot be identified, even if the personally-identifying information is viewed, the personal information can be prevented from being leaked.

Additionally, when the ultrasonic imaging diagnostic apparatus 101 is carried outside the healthcare center and is leaked, because the personal information cannot be identified, even if the personally-identifying information is viewed, the personal information can be prevented from being leaked.

Additionally, according to input, the personal information, the personally-identifying information, or the examination information can be displayed on the display 14 when the ultrasonic imaging diagnostic apparatus 101 is in the healthcare center.

According to the above-described embodiments, the system can prevent the personal information from being leaked and the medical staff can take the medical imaging diagnosis apparatus from the healthcare center at ease, thus improving convenience.

In this embodiment, when the examination information of an examination already executed for a specific patient is taken outside the healthcare center for a home visit to the specific patient, an example of using a small ultrasonic imaging diagnostic apparatus is described. But this embodiment is not limited and can be applied to an apparatus that transmits and receives the data through the network 400 and stores the data, for example, a laptop personal computer, mobile phone, or portable terminal for reading an electronic book.

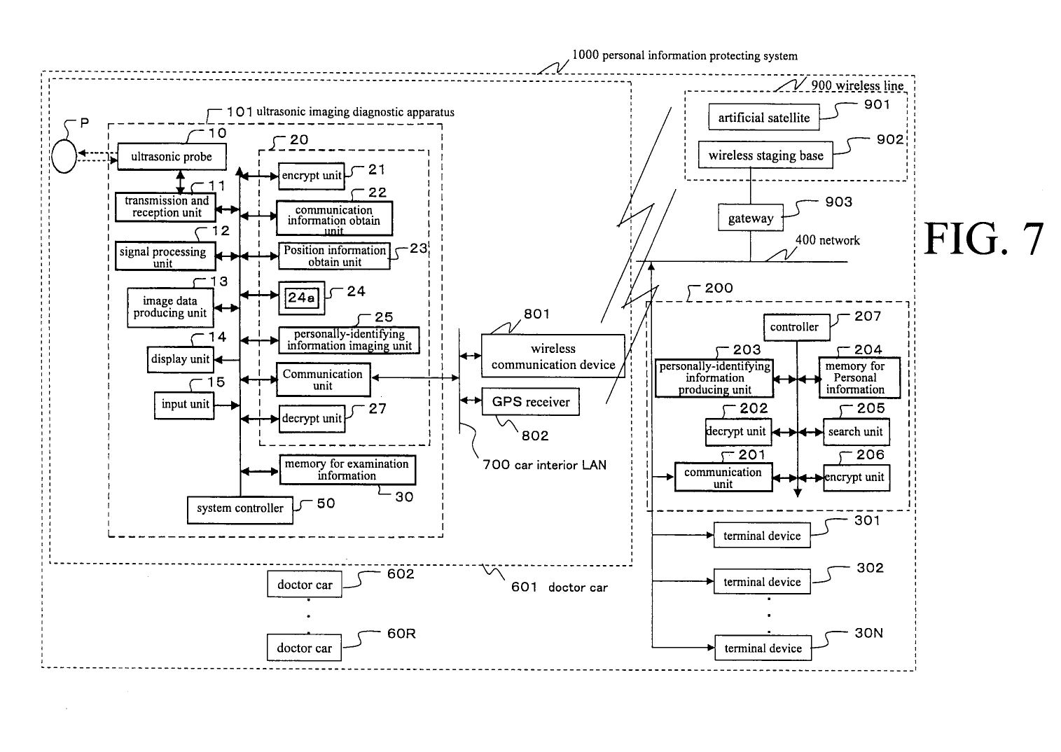

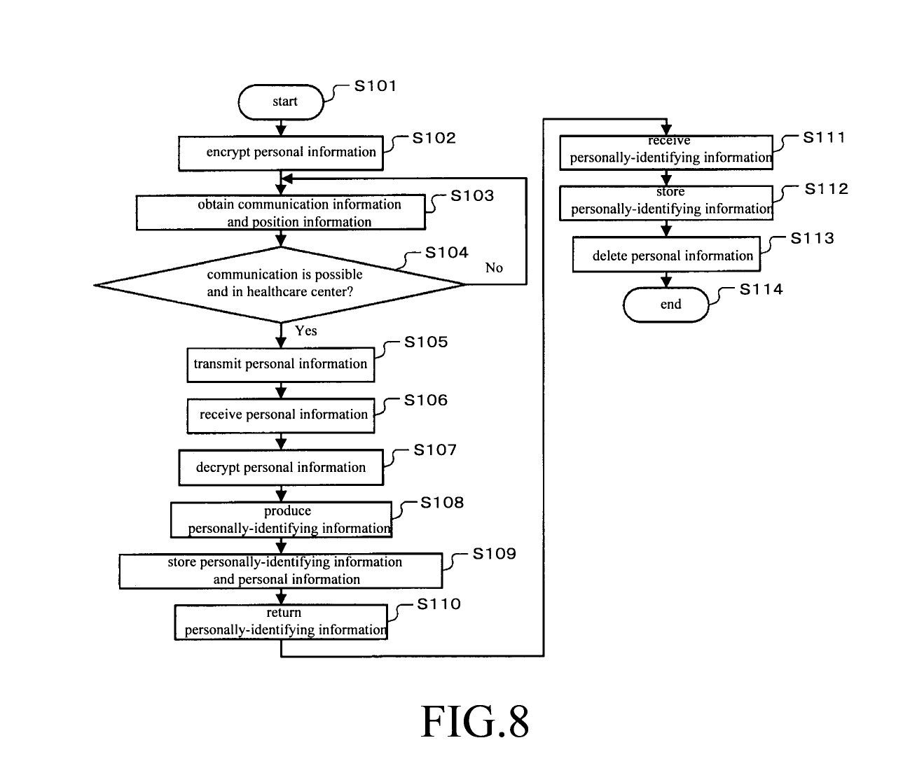

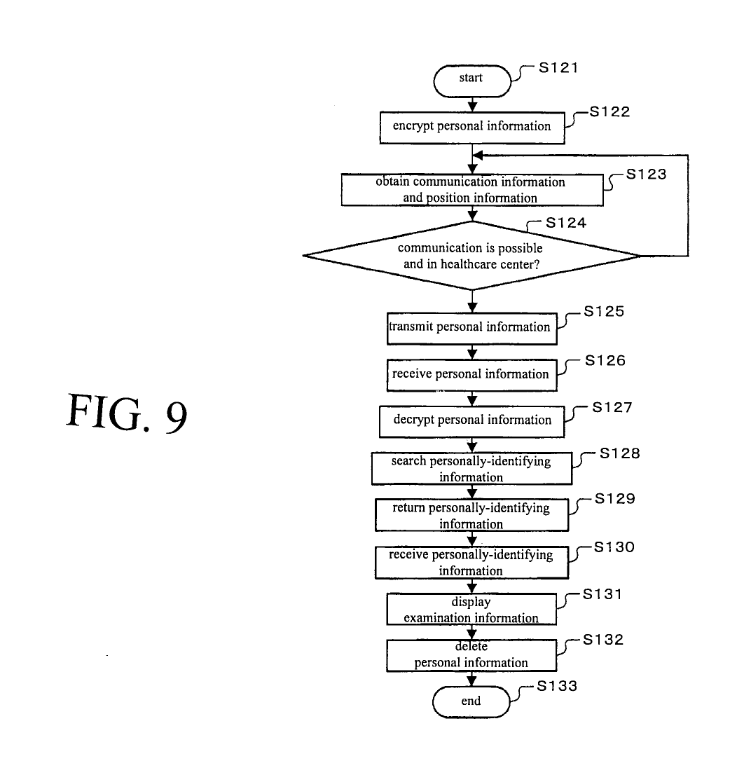

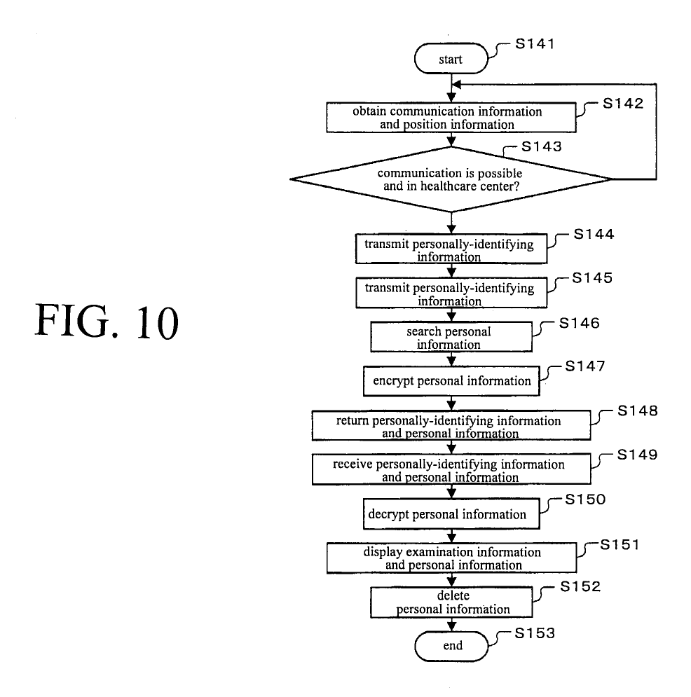

Hereinafter, an embodiment will be described of a personal information protecting system that connects a doctor car that includes an ultrasonic imaging diagnostic apparatus, an X-ray diagnosis apparatus, or an X-ray computed tomography apparatus to the personal information managing server in the healthcare center, with reference to FIG. 7, FIG. 8, FIG. 9, and FIG. 10.

FIG. 7 is a block diagram showing a structure of a personal information protecting system 1000 according to a second embodiment.

The personal information protecting system 1000 has one or more doctor cars 601-60R for picking up and transporting an emergency patient to the healthcare center, and for transporting the medical imaging diagnosis apparatus.

The personal information protecting system 1000 has a personal information managing server 200. The personal information managing server 200 connects to the medical image diagnostic apparatus on the doctor car 601-60R through the network 400, and makes personally-identifying information for identifying the personal information, for example, the name of the subject P, birth date, sex, etc. and stores the personal information.

In addition, the personal information protecting system 1000 has terminal devices 301-30N. Each terminal device 301-30N is arranged in the healthcare center, connects to the medical imaging diagnosis apparatus and has an inputting unit for inputting the personal information of the subject P for accepting the examination by the medical imaging diagnosis apparatus.

At first, the doctor car 601 will be described. Each doctor car 602-60R is similar to the doctor car 601, and the description of these apparatuses will be skipped.

As shown in FIG. 7, the doctor car 601 includes an ultrasonic imaging diagnostic apparatus 101, a wireless communication device 801, and a GPS receiver 802 to detect the position of the doctor car 601.

In terms of the composition of the ultrasonic imaging diagnostic apparatus 101 on the doctor car 601, the communication information obtain unit 22, the position information obtain unit 23, and the communication unit 26 that are different from those described above and will be described, but the description of other units of the ultrasonic imaging diagnostic apparatus 101 will be skipped.

The communication information obtain unit 22 obtains the communication information for judging whether the medical imaging diagnosis apparatus can communicate with the personal information managing server 200 through the wireless communication device 801 and the wireless line 900.

The position information obtain unit 23 obtains the position information of the ultrasonic imaging diagnostic apparatus 101 measured by the GPS receiver 802.

In the position information obtain unit 23, the position information can be obtained by RFID (Radio Frequency Identification) or wireless LAN (Local Area Network), as with the first embodiment as described above.

The communication unit 26 connects a car interior LAN 700 of the doctor car 601, and communicates via the wireless line 900 comprising the artificial satellite 901 or the wireless staging base 902 through the wireless communication device 801 in the doctor car 601.

The communication unit 26 receives the personal information through the wireless line 900 that is input by a terminal device 301-30N at the start of the examination transmitted through the network 400, and outputs the received personal information to the encrypt unit 21.

The communication unit 26 transmits the personal information encrypted by the encrypt unit 21 and receives the personally-identifying information returned through the network 400 by the personal information managing server 200, in response to the transmission of the personal information.

Additionally, the communication unit 26 transmits the personally-identifying information input by the input unit 15 and stored in the memory for examination information 30 to the personal information managing server 200 through the network 400, and receives the encrypted personal information returned through the network 400 by the personal information managing server 200, in response to the transmission of the personally-identifying information.

The wireless line 900 connects the network, for example, a public line or private line, through the gateway 903 for communicating using a communication protocol, and herewith many kinds of information communication based on standardized communication protocols, for example, TCP/IP can be used.

In this embodiment, the medical imaging diagnosis apparatus on the doctor car 601-60R is an ultrasonic imaging diagnostic apparatus, but this embodiment is not limited to an ultrasonic imaging apparatus. For example, this embodiment can be applied to a compact X-ray diagnosis apparatus or an X-ray computed tomography apparatus.

Hereinafter, an example of a method executed by the personal information protecting system 1000 will be described with reference to FIG. 7 , FIG. 8, FIG. 9, and FIG. 10.

FIG. 8 is a flowchart showing a process executed by the personal information protecting system 1000 during the examination.

FIG. 9 and FIG. 10 are flowcharts showing a process executed by the personal information protecting system 1000 for outputting the examination information.

FIG. 9 is a flowchart showing a process executed by the personal information protecting system 1000 for outputting the examination information in response to the input of the personal information, while FIG. 10 is a flowchart showing a process executed by the personal information protecting system 1000 for outputting the examination information in response to the input of the personally-identifying information.

At first, the process executed by the personal information protecting system 1000 during the examination will be described.

In FIG. 8, for the examination of the subject P using the ultrasonic imaging diagnostic apparatus 101, when the personal information of the subject P is input by the input unit 15 of the ultrasonic imaging diagnostic apparatus 101 in step 5101, the personal information protecting system 1000 starts the examination process.

The system controller 50 of the ultrasonic imaging diagnostic apparatus 101 gives an order to start the examination to the memory for examination information 30.

In step S102, the encrypt unit 21 of the personal information processing unit 20 encrypts the personal information input by the input unit 15 and outputs the encrypt personal information to the judge unit 24.

In step S103, the communication information obtain unit 22 and the communication information taking unit 23 obtain the communication information and the position information at constant time intervals and output the communication information and the position information to the judge unit 24.

After the judge unit 24 stores the personal information output from the encrypt unit 21 in the memory 24a, the judge unit 24 judges whether the personal information stored in the memory 24a can be sent to the personal information managing server 200 through the wireless line 900 and the network 400 based on the communication information output from the communication information obtain unit 22 and the position information obtain unit 23.

When the GPS receiver 802 is used, the position information obtain unit 23 stores the data of the area when the doctor car 601 can transmit the personal information to the personal information managing server 200 and the judge unit 24 judges whether the personal information stored in the memory 24a can be sent from the doctor car 601 to the personal information managing server 200 through the wireless line 900 and the network 400, based on the data of the area when the doctor car 601 can transmit the personal information to the personal information managing server 200 and the position information of the doctor car 601 transmitted to the GPS receiver 802.

When RFID and wireless LAN is used, the judge unit 24 judges that the ultrasonic imaging diagnostic apparatus 101 is outside the healthcare center when the access point is not found in the healthcare center.

Further, when the ultrasonic imaging diagnostic apparatus 101 can communicate with the personal information managing server 200 and the ultrasonic imaging diagnostic apparatus 101 (the doctor car 601) is in the healthcare center (Yes in Step S104), the process proceeds to Step S105.

When the ultrasonic imaging diagnostic apparatus 101 cannot communicate with the personal information managing server 200 or the ultrasonic imaging diagnostic apparatus 101 (the doctor car 601) is outside the healthcare center (No in Step S104), the process returns to Step S103.

When the ultrasonic imaging diagnostic apparatus 101 (the doctor car 601) moves from outside the healthcare center to inside the healthcare center, the ultrasonic imaging diagnostic apparatus 101 tries to connect to the network 400, and when the ultrasonic imaging diagnostic apparatus 101 can communicate with the personal information managing server 200, the process moves to Step S105.

After “Yes in Step S104”, the judge unit 24 stores the personal information in the memory 24a and outputs the personal information to the communication unit 26.

In step S105, the communication unit 26 transmits the personal information output by the judge unit 24 to the personal information managing server 200 through the wireless line 900 and the network 400 by using the wireless communication device 801.

In step S106, the communication unit 201 of the personal information managing server 200 receives the personal information transmitted by the ultrasonic imaging diagnostic apparatus 101 and outputs the received personal information to the decrypt unit 202.

In step S107, the decrypt unit 202 decrypts the personal information output by the communication unit 201, and outputs the decrypted personal information to the personally-identifying information producing unit 203.

In step S108, the personally-identifying information producing unit 203 produces the personally-identifying information to identify the personal information output by the decrypt unit 202 and outputs the personally-identifying information to the communication unit 201, and outputs the personally-identifying information and the personal information decrypted by the decrypt unit 202 and identified by the personally-identifying information.

In step S109, the memory for personal information 204 stores the personally-identifying information and the personal information output by the personally-identifying information producing unit 203.

Thus, leaking of the personal information from the ultrasonic imaging diagnostic apparatus 101 inside and outside the healthcare center can be prevented by storing the personal information input by the input unit 15 to the personal information managing server 200, which is outside the ultrasonic imaging diagnostic apparatus 101.

Further, when the ultrasonic imaging diagnostic apparatus 101 is carried outside the healthcare center, the system can prevent the personal information from being publicly exposed and being leaked.

Further, when the ultrasonic imaging diagnostic apparatus 101 is carried outside the healthcare center and is stolen, the system can prevent the personal information from being viewed and being leaked.

In step S110, the communication unit 201 returns the personally-identifying information output by the personally-identifying information producing unit 203 to the wireless communication device 801 of the doctor car 601 through the network 400 and to the ultrasonic imaging diagnostic apparatus 101.

In step S111, the communication unit 26 of the ultrasonic imaging diagnostic apparatus 101 receives the personally-identifying information returned from the personal information managing server 200 through the network 400 and outputs the received personally-identifying information to the personally-identifying information managing unit 25.

When the personal information is input by the input unit 15 and the operation for starting the examination of the subject P that has the personal information begins, the signal processing unit 12 processes the received signal output by the transmission and reception unit 11 and produces the B-mode data and outputs the B-mode data to the image data producing unit 13.

The image data producing unit 13 produces the B-mode image data based on the B-mode data output by the signal processing unit 12, and the display unit 14 displays the B-mode image data in real time.

Further, when the display unit 14 displays the B-mode image data that is helpful for the examination and a stop operation is executed by the input unit 15, the image data producing unit 13 stores the examination information including the B-mode image data to the memory for examination information 30.

The personally-identifying information managing unit 25 displays the personally-identifying information output by the communication unit 26 on the display unit 14.

In step S112, the communication unit 26 stores the personally-identifying information related to the examination information of the subject P that has the personal information that is identified by the personally-identifying information.

When the ultrasonic imaging diagnostic apparatus 101 is carried outside the healthcare center and is used in the examination, and after the examination the ultrasonic imaging diagnostic apparatus 101 is returned inside the healthcare center, after the examination information is stored in the ultrasonic imaging diagnostic apparatus 101, the display unit 14 displays the examination information output by the communication unit 26 and the personally-identifying information is stored in relation to the examination information in the memory for examination information 30.

Further, when the ultrasonic imaging diagnostic apparatus 101 is used in the examination, after the personally-identifying information output by the communication unit 26 is stored in the memory of the ultrasonic imaging diagnostic apparatus 101, the examination information of the subject P that has the personal information identified by the personally-identifying information and the personally-identifying information stored related to the stored examination information in the memory is stored.

Thus, the examination information is stored in the ultrasonic imaging diagnostic apparatus 101 and the examination information can be identified by storing the personally-identifying information related to the examination information.

Further, when the ultrasonic imaging diagnostic apparatus 101 is carried outside the healthcare center, because the personal information cannot be identified, even if the personally-identifying information is viewed, the personal information can be prevented from being leaked.

Additionally, when the ultrasonic imaging diagnostic apparatus 101 is carried outside the healthcare center and is leaked, because the personal information cannot be identified, even if the personally-identifying information is viewed, the personal information can be prevented from being leaked.

In step S113, after the personally-identifying information is stored, the personal information stored in the memory 24a is deleted.

Thus, after the personally-identifying information that identifies the personal information and was produced by the personal information managing server 200 is stored in the memory 24a for examination information, the personal information can be prevented from being viewed by an outsider, except the operator, despite being inside and outside the healthcare center and leaked.

In step S114, after the personal information is deleted, the personal information protecting system 1000 stops the examination.

Next, a method executed by the personal information protecting system 1000 for outputting the examination information in response to the input of the personal information is described.

FIG. 9 is a flowchart showing a process executed by the personal information protecting system 1000 for outputting the examination information in response to the input of the personal information in the examination.

In step S121, after the examination information and the personally-identifying information are stored by the memory for examination information 30 in the ultrasonic imaging diagnostic apparatus 101, when the personal information of the subject P is input by the input unit 15 of the ultrasonic imaging diagnostic apparatus 101, the personal information protecting system 1000 starts the examination process.

The system controller 50 of the ultrasonic imaging diagnostic apparatus 101 gives an order to output the examination information to the display unit 14, the personal information processing unit 20, and the memory for examination information 30.

In step S122, the encrypt unit 21 of the personal information processing unit 20 encrypts the personal information input by the input unit 15 and outputs the encrypt information to the judge unit 24.

In step S123, the communication information obtain unit 22 and the position information obtain unit 23 obtain the communication information and the position information at constant time intervals and output the communication information and the position information to the judge unit 24.

After the judge unit 24 stores the personal information output from the encrypt unit 21 in the memory 24a, the judge unit 24 judges whether the personal information output by the encrypt unit 21 can be sent to the personal information managing server 200 based on the communication information output from the communication information obtain unit 22 and the position information obtain unit 23.

The method used to judge by the judge unit 24 in this situation is similar to the one shown in the flowchart of FIG. 8.

When the ultrasonic imaging diagnostic apparatus 101 can communicate with the personal information managing server 200 and the ultrasonic imaging diagnostic apparatus 101 (the doctor car 601) is in the healthcare center (Yes in Step S124), the process proceeds to Step S125.

Further, when the ultrasonic imaging diagnostic apparatus 101 cannot communicate with the personal information managing server 200 or the ultrasonic imaging diagnostic apparatus 101 (the doctor car 601) is outside the healthcare center (No in Step S124), the process returns to Step S123.

After Step S124, the judge unit 24 stores the personal information and outputs the personal information to the communication unit 26.

In step S125, the communication unit 26 transmits the personal information output by the judge unit 24 to the personal information managing server 200 through the wireless line 900 and the network 400 by using the wireless communication device 801.

In step S126, the communication unit 201 of the personal information managing server 200 receives the personal information transmitted by the ultrasonic imaging diagnostic apparatus 101 through the network 400.

The communication unit 201 outputs the personal information received by the ultrasonic imaging diagnostic apparatus 101.

In step S127, the decrypt unit 202 decrypts the personal information received by the communication unit 201 and outputs the decrypted personal information to the search unit 205.

In step S128, the search unit 205 searches the personally-identifying information related to an personal information output by the decrypt unit 202 in the personally-identifying information and the personal information stored by the memory for personal information 204 and outputs the searched personally-identifying information.

In step S129, the communication unit 201 returns the personally-identifying information output by the search unit 205 to the wireless communication device 801 through the wireless line 900 and the network 400 to the ultrasonic imaging diagnostic apparatus 101.

In step S130, the communication unit 26 of the ultrasonic imaging diagnostic apparatus 101 receives the personally-identifying information returned from the personal information managing server 200 through the network 400, and outputs the received personally-identifying information to the personally-identifying information managing unit 25.

In step S131, the personally-identifying information managing unit 25 reads the examination information related to the personally-identifying information and output by communication unit 26, and displays the read examination information.

In step S132, after the viewing of the examination information displayed by the display unit 14, when an operation of ending viewing is executed by the input unit 15, the judge unit 24 deletes the personal information stored in the memory 24a.

Thus, after the personally-identifying information that identifies the personal information and was produced by the personal information managing server 200 is stored in the ultrasonic imaging diagnostic apparatus 101, the personal information can be prevented from being viewed by an outsider, except the operator, despite being inside and outside the healthcare center and leaked.

In step S133, when the system controller 50 gives an order to stop transmission to the display unit 14, the personal information processing unit 20 and the memory for examination information 30, the personal information protecting system 1000 stops the examination.

Thus, the examination information can be output by inputting the personally-identifying information, except for inputting the personal information.

In what follows, a process executed by the personal information protecting system 1000 when the examination information is output in response to inputting the personally-identifying information is described.

FIG. 10 is a flowchart showing a process executed by the personal information protecting system 1000 for outputting the examination information in response to the input of the personally-identifying information during the examination.

In Step S112 in FIG. 8, when the personally-identifying information stored in the memory for examination information 30 is displayed on the display 14, for example, the personally-identifying information for identifying the personal information of the patient is recorded in the medical record.

In step S141, after the examination information and the personally-identifying information are stored in the memory for examination information 30 of the ultrasonic imaging diagnostic apparatus 101, when the personally-identifying information is input by the input unit 15, the personal information protecting system 1000 starts to output the examination information.

The display 14 displays a view of the personally-identifying information stored in the memory for examination information 30, and one of the personally-identifying information items is selected by the input unit 15 in the view displayed.

The system controller 50 of the ultrasonic imaging diagnostic apparatus 101 gives an order to output the examination information to the display unit 14, the personal information processing unit 20, and the memory for examination information 30.

In step S142, the communication information obtain unit 22 and the communication information taking unit 23 obtain the communication information and the position information at constant time intervals and output the communication information and the position information to the judge unit 24.

After the personally-identifying information input by the input unit 15 is stored in the memory 24a, the judge unit 24 judges whether the personally-identifying information input can be sent to the personal information managing server 200, based on the communication information output from the communication information obtain unit 22 and the position information output from the position information obtain unit 23.

The method used to judge by the judge unit 24 in this situation is similar to the one shown in the flowchart of FIG. 8.

Further, when the ultrasonic imaging diagnostic apparatus 101 can communicate with the personal information managing server 200 and the ultrasonic imaging diagnostic apparatus 101 (the doctor car 601) is in the healthcare center (Yes in Step S143), the process proceeds to Step S144.

When the ultrasonic imaging diagnostic apparatus 101 (the doctor car 601) cannot communicate with the personal information managing server 200 or the ultrasonic imaging diagnostic apparatus 101 is outside the healthcare center (No in Step S143), the process returns to Step S142.

After “Yes in Step S143”, the judge unit 24 stores the personally-identifying information in the memory 24a and outputs the personally-identifying information to the communication unit 26.

In step S144, the communication unit 26 transmits the personally-identifying information output by the judge unit 24 to the personal information managing server 200 through the network 400.

In step S145, the communication unit 201 of the personal information managing server 200 receives the personally-identifying information transmitted by the ultrasonic imaging diagnostic apparatus 101 through the wireless line 900 and the network 400 by using the wireless communication device 801.

The communication unit 201 outputs the personally-identifying information received by the ultrasonic imaging diagnostic apparatus 101.

In step S146, the search unit 205 searches the personal information related to the personally-identifying information output by the communication unit 201 in the personally-identifying information and the personal information stored by the memory for personal information 204, and outputs the searched personal information and the personally-identifying information related to the personal information to the encrypt unit 206.

In step S147, the encrypt unit 206 encrypts the personal information output by the search unit 205 and outputs the encrypted personal information and the personally-identifying information related to the personal information to the communication unit 201.

In step S148, the communication unit 201 returns the encrypted personal information and the personally-identifying information output by the encrypt unit 206 to the wireless communication device 801 of the doctor car 601 and to the ultrasonic imaging diagnostic apparatus 101.

In step S149, the communication unit 26 of the ultrasonic imaging diagnostic apparatus 101 receives the personal information and the personally-identifying information returned from the personal information managing server 200 through the network 400, and outputs the received personal information and the personally-identifying information to the decrypt unit 27.

In step S150, the decrypt unit 27 decrypts the personal information output by the communication unit 26, and outputs the decrypted personal information and the personal information related the personal information to the personally-identifying information managing unit 25.

In step S151, the personally-identifying information managing unit 25 reads the examination information related to the personally-identifying information output from the decrypt unit 27 and displays the read examination information.

In step S152, after the viewing of the examination information and the personal information displayed by the display unit 14, when an operation of ending the viewing is executed by the input unit 15, the judge unit 24 deletes the personally-identifying information stored in the memory 24a.

In step S153, when the system controller 50 gives an order to stop transmission to the display unit 14, the personal information processing unit 20 and the memory for examination information 30, the personal information protecting system 1000 stops the examination.

According to the embodiment described above, the personal information for the examination input by each terminal device 301-30N or the input unit 15 can be stored in the personal information managing server 200 outside the ultrasonic imaging diagnostic apparatus 101.

Herewith when the ultrasonic imaging diagnostic apparatus 101 (the doctor car 601) is carried outside the healthcare center, the system can prevent the personal information from being publicly exposed and being leaked.

Further, when the ultrasonic imaging diagnostic apparatus 101 is carried outside the healthcare center and is leaked, personal information can be prevented from being viewed and leaked.

From the time the memory for examination information 30 stores the personally-identifying information produced in the personal information managing server 200 for identifying the personal information, the personal information input for the examination can be stored encrypted in the ultrasonic imaging diagnostic apparatus 101.

Herewith when the ultrasonic imaging diagnostic apparatus 101 is carried outside the healthcare center, the system can prevent the personal information from being publicly exposed and being leaked.

Additionally, the personal information can be related to the examination information and be stored with the examination information in the ultrasonic imaging diagnostic apparatus 101.

Herewith the examination information is stored in the ultrasonic imaging diagnostic apparatus 101 and the examination information can be identified by storing the personally-identifying information related to the examination information.

Further, when the ultrasonic imaging diagnostic apparatus 101 is carried outside the healthcare center, because the personally-identifying information cannot be identified, even if the personally-identifying information is viewed, the system can prevent the personal information from being leaked.

Further, when the ultrasonic imaging diagnostic apparatus 101 is carried outside the healthcare center and is stolen, because the personally-identifying information cannot be identified, even if the personally-identifying information is viewed, the system can prevent the personal information from being viewed and being leaked.

Additionally, according to input of the personal information or the personally-identifying information, the examination information or the personal information can be displayed on the display 14 when the ultrasonic imaging diagnostic apparatus 101 (the doctor car 601) is in the healthcare center.

Herewith when the ultrasonic imaging diagnostic apparatus 101 is carried outside the healthcare center, the system can prevent the personal information from being in the public exposure and being leaked.

According to the composition above mentioned, the system can prevent the personal information from being leaked and the medical staff can take the medical imaging diagnosis apparatus from the healthcare center at ease with improved convenience.

While certain embodiments have been described, these embodiments have been presented by way of example only, and are not intended to limit the scope of the inventions. Indeed the novel methods and systems described herein may be embodied in a variety of other forms; furthermore, various omissions, substitutions, and changes in the form of the methods and systems described herein may be made without departing from the spirit of the inventions. The accompanying claims and their equivalents are intended to cover such forms or modifications as would fall within the scope and spirit of the inventions.

What is claimed is:

1. A medical image diagnostic apparatus producing image data of an examination subject, comprising:
an input unit to input personal information of the subject;
a judge unit to judge whether the personal information can be transmitted to a personal information managing server that stores the personal information;
a communication unit to transmit the personal information to the personal information managing server and to receive personally-identifying information that identifies the personal information, when the judge unit judges that the personal information can be transmitted to the personal information managing server; and
an examination information memory to store the personally-identifying information.

2. The medical image diagnostic apparatus according to claim 1, wherein the judge unit is configured to judge whether the personal information can be transmitted based on position information obtained by a position information obtaining unit.

3. The medical image diagnostic apparatus according to claim 2, wherein the judge unit is configured to judge that the personal information can be transmitted when the medical image diagnostic apparatus is in a healthcare center, and is configured to judge that the personal information can not be transmitted when the medical image diagnostic apparatus is outside the healthcare center.

4. The medical image diagnostic apparatus according to claim 1, further comprising an encrypt unit to encrypt the personal information,
wherein the communication unit is configured to transmit the encrypted personal information encrypted by the encrypt unit to the personal information managing server.

5. The medical image diagnostic apparatus according to claim 4, further comprising:
a personal information memory to store the personal information encrypted by the encrypt unit; and
a delete unit configured to delete the personal information,
wherein the personally-identifying information is stored by the examination information memory.

6. The medical image diagnostic apparatus according to claim 4, further comprising:
a personally-identifying managing unit to read the examination information related to the personally-identifying information to identify the personal information returned by the personal information managing server, in response to the personal information transmitted to the personal information managing server; and
a display unit to display the examination information.

7. The medical image diagnostic apparatus according to claim 1, wherein the input unit is configured to input the personally-identifying information stored by the examination information memory,
the medical image diagnostic apparatus further comprising:
a decrypt unit to decrypt the encrypted personal information identified by the personally-identifying information returned from the personal information managing server in response to the personally-identifying information transmitted;
a personally-identifying information managing unit to read the examination information related to the decrypted personal information, and the personally-identifying information for identifying the personal information; and
a display unit to display the examination information read by the personally-identifying information managing unit and the decrypted personal information.

8. The medical image diagnostic apparatus according to claim 1,
wherein when the personal information of the subject is input by the input unit, the judge unit is configured to judge whether the personal information can be transmitted to the personal information managing server; and
when the judge unit judges that the personal information input by the input unit can be transmitted to the personal information managing server, the communication unit receives the personally-identifying information to identify the personal information returned by the personal information managing server in response to the personal information transmitted,
wherein the medical image diagnostic apparatus further comprises:
a transferring unit to transfer the examination information related to the personally-identifying information received by the communication unit with the personally-identifying information to the personal information managing server.

9. The medical image diagnostic apparatus according to claim 8,
wherein when the personal information of the subject is input by the input unit, the judge unit judges whether the personal information can be transmitted to the personal information managing server;
when the judge unit judges that the personal information can be transmitted, the communication unit transmits the personal information to the personal information managing server and receives the personally-identifying information and the examination information related to the personally-identifying information in response to the transmission; and
the examination information memory stores the personally-identifying information and the examination information.

10. A medical image display device for displaying medical information of an examination subject, comprising:
an input unit to input personal information of the subject;
a judge unit to judge whether the personal information can be transmitted to a personal information managing server that stores the personal information;
a communication unit to transmit the personal information to the personal information managing server and to receive personally-identifying information that identifies the personal information when the judge unit judges that the personal information can be transmitted to the personal information managing server;
an examination information memory to store the personally-identifying information; and
a transferring unit to transfer the examination information related to the personally-identifying information received by the communication unit with the personally-identifying information to the personal information managing server,
wherein when the personal information is input by the input unit, the judge unit judges whether the personal information can be transmitted to the personal information managing server; and
when the judge unit judges when the personal information can be transmitted, the communication unit transmits the personal information to the personal information managing server and receives the personally-identifying information.

11. The medical image display device according to claim 10,
wherein when the personal information of the subject is input by the input unit, the judge unit judges whether the personal information can be transmitted to the personal information managing server;
when the judge unit judges that the personal information input by the input unit can be transmitted to the personal information managing server, the communication unit receives the personally-identifying information to identify the personal information returned by the personal information managing server in response to the personal information transmitted; and
the examination information memory stores the examination information.

12. A personal information protecting system having a medical image diagnostic apparatus producing image data of a subject under examination and a personal information managing server for accessing the medical image diagnostic apparatus through a network and storing the personal information,
the medical image diagnostic apparatus comprising:
a input unit to input personal information of the subject;
a judge unit configured to judge whether the personal information can be transmitted to the personal information managing server that stores the personal information;
a communication unit to transmit the personal information to the personal information managing server and to receive personally-identifying information that identifies the personal information when the judge unit judges that the personal information can be transmitted to the personal information managing server; and
an examination information memory to store the personally-identifying information,
wherein the personal information managing server comprises:
a personally-identifying information producing unit configured to produce the personally-identifying information;
a personal information memory to store the personally-identifying information and the personal information identified by the personally-identifying information; and
a communication unit configured to receive the personal information transmitted by the medical image diagnostic apparatus through the network, and to return the personally-identifying information, produced by the personally-identifying information producing unit in response to reception of the personal information, to the medical image diagnostic apparatus through the network.
